(19) United States
(12) Patent Application Publication
(10) Pub. No.: US 2012/0302897 A1
(43) Pub. Date: Nov. 29, 2012

(54) HEART RATE VARIABILITY DEVICE AND CLOUD HEALTH MANAGEMENT SYSTEM

(75) Inventors: Danny Chen, Taipei City (TW); Earl Chen, Tao Yuan Hsien (TW)

(73) Assignee: VICON HEALTHCARE INTERNATIONAL INC., New Taipei City (TW)

(21) Appl. No.: 13/114,299

(22) Filed: May 24, 2011

(51) Int. Cl. A61B 5/02; G06Q 50/22

(52) U.S. Cl. 600/481; 705/2

(57) ABSTRACT

The present invention relates to a heart rate variability device and cloud health management system. The heart rate variability device comprises a CPU and is adapted with a telecommunication and communication module. The cloud health management system comprises more than one cloud server and a stress-relieved mechanism server. The invention may serve as a heart rate variability device on one hand, and on the other hand may make use of the telecommunication and communication module to internet-connecting to a hospital, a stress-relieved mechanism and a cloud, thereby forming a new business model to achieve the objectives of reducing cost for heart rate variability disease and accomplishing entire health management.

### BACKGROUND OF THE INVENTION

1. Field of the Invention

The invention relates to a heart rate variability device and cloud health management system, in particular to one that may serve as a heart rate variability device on one hand, and on the other hand it can also use telecommunications and communication module to be internet-connected to a hospital, a stress-relieved mechanism and a cloud, thereby forming a new business model to reduce cost for heart rate variability disease and to accomplish entire health management.

2. Description of Related Art

Traditionally, the so-called autonomic computer device, which is directed to providing an analysis method for obtaining inspection data of the function of an autonomic nerve system using a non-invasive approach, and to a computer device thereof, such as the so-called yin and yang instrument. In theory, it is observed by Task Force of European Society of Cardiology and the North American Society of Pacing and Electrophysiology as well as Malliani, etc. that heart rate variability is affected by fluctuations by respiration, and in addition, responds function of the autonomic nerve system function. The so-called heart rate variability refers to the rate of heart beating rate (i.e. heart rate), in addition to still maintain 60-90 times per minute, while hiding therein some rules and irregular fluctuations. As the magnitude of these fluctuations is too small in amplitude, the traditional analysis methods can not be accurate analysis. Till recently, techniques of signal detection and processing are in progress tremendously. Thus, with the aid of spectrum analysis, the researchers found the minor fluctuations in the heart rate variability may be divided into two groups, namely, one being high frequency (HF) variability and the other being low frequency (LF) variability. The low frequency (LF) variability may further be divided into low frequency variability and extremely low frequency variability. The high frequency part is synchronously with the breathing signal of the human being and is also called a breathing component, namely about one time per 3 seconds for the human being. It is unclear as to where the low frequency part comes. The scholars infer that it may be correlated with blood vessel movement or the feeling pressing the reflection, namely, about one time per 10 seconds. At present, many physiologists and cardiology doctors agree that the HF heart rate variability or total power (TP) represents the function of the parasympathetic nerve, the LF heart rate variability represents the entire activity of the autonomic nerve, and the ratio of the LF variability and the HF variability (LF/HF) is able to reflect the activity of the sympathetic nerve.

It is also found in the research that except serving as an autonomic nerve index, the heart rate variability is capable of reflecting a variety of body information. For example, the heart rate variability will be decreased for a patient having intracranial pressure. Not very long ago, an investigation from Bramingham, U.S.A. reveals that if the LF part of the heart rate of an old man is lower than a standard difference, the probability of dying is 1.7 times of that of a normal people.

Though a non-invasive heart rate variability device (such as a yin and yang instrument) has been developed, due to that the heart rate variability device is very expensive, it is unable to make use of the cloud operation for economical and effective use and it is unable for a pressure patient to utilize a telemedicine network for pressure diagnosis and treatment. It is indeed a need to develop a new heart rate variability device capable of serving as a heart rate variability device on one hand, and on the other hand capable of making use of a telecommunication and communication module to be internet-connected to a hospital, a stress-relieved mechanism and a cloud, thereby forming a new business model to reduce cost for heart rate variability disease and to accomplish entire health management.

### SUMMARY OF THE INVENTION

The present invention is to provide a heart rate variability device, comprising:

a sensing element for sensing a heart beat signal of a pressure patient;

a signal processing module having a first high pass filter, a first amplifier, a first low pass filter, a voltage/current converter, a comparator circuit, a second high pass filter and an analog/digital converter, for converting the heart beat signal into an autonomic nerve output signal;

a heart rate variability central processing unit (CPU), being installed with a heart rate variability processing software for outputting inspection data after processing and operating the autonomic nerve output signal; and

a wireless telecommunication and communication module for receiving the inspection data and transferring the inspection data to an internet access device via wireless communication.

According to the invention, the internet access device is a base station or a PC.

According to the invention, the wireless telecommunication and communication module adopts communication standard of 2.75G, 3G, 3.5G or 4G.

The invention further provides a cloud health managing system of heart rate variability frequency spectrum, comprising:

more than one inspection data input/output device for inputting or outputting more than one inspection data of a pressure patient;

more than one internet access device, being connected to the inspection data input/output device for transferring the inspection data to a cloud via the internet access device;

more than one cloud server, being connected to the internet access device for storing the inspection data, and making use of the inspection data input/output device for accessing the inspection data; and

a stress-relieved mechanism server, being connected to the cloud server and having a search engine, in which as a pressure index in the inspection data exceeds a preset value, a hyperlink of a stress-relieved mechanism is presented on the inspection data input/output device so as to connect to a website of the stress-relieved mechanism to facilitate proceeding of stress-relieving of the pressure patient.

According to the invention, the inspection data input/output device is preferably a computer or a heart rate variability device as mentioned above.

According to the invention, the stress-relieved mechanism is preferably a stress-relieved music mechanism, yoga mechanism, imperial sacrifices sit mechanism, mind traveling mechanism, hot spring malingers mechanism, game software mechanism, Chinese shadow boxing mechanism, forward ponder learning mechanism, essential oil stress-relieved mechanism, stress-relieved massager mechanism, meditation courses mechanism, rhythm breathing mechanism, Brocade qigong mechanism or stress-relieved food and drink restaurant.

### BRIEF DESCRIPTION OF THE DRAWINGS

FIG. 1 shows a circuit block diagram of a heart rate variability device according to a preferred embodiment of the invention;

FIG. 2 is a flow chart showing analysis steps for an autonomic nerve according to a preferred embodiment of the invention;

FIG. 3 is a flow chart showing steps for converting from a time domain to a frequency domain with respect to a heart beat signal according to a preferred embodiment of the invention;

FIG. 4 is a flow chart showing steps to search corresponding analysis data in a built-in tablet according to a preferred embodiment of the invention;

FIG. 5 is a schematic diagram showing six pressure statuses presented on a display of the heart rate variability device of the invention;

FIG. 6 shows a schematic block diagram of a cloud health management system for heart rate variability according to a preferred embodiment of the invention;

FIG. 7 is a schematic diagram in front view of the heart rate variability device according to the preferred embodiment of the invention.

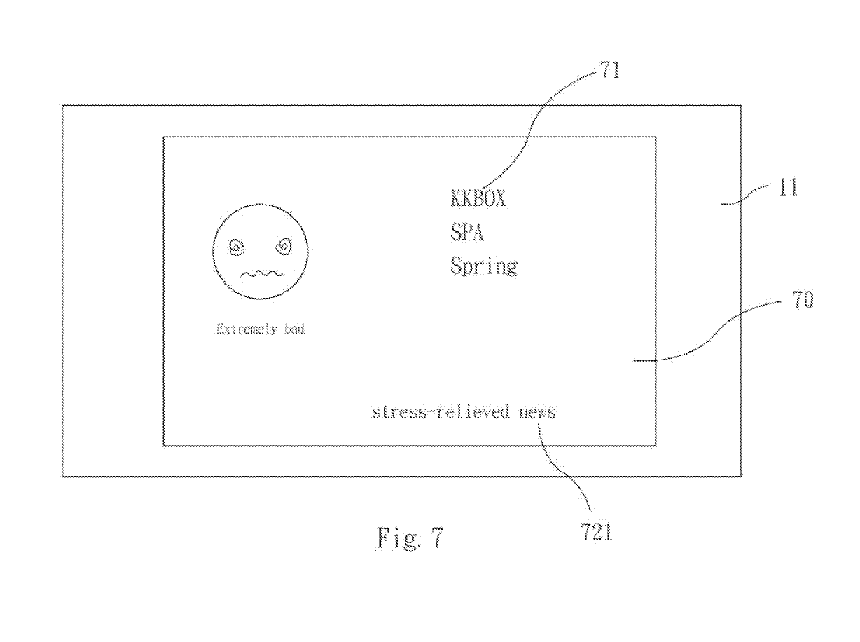

### DETAILED DESCRIPTION OF THE PREFERRED EMBODIMENT

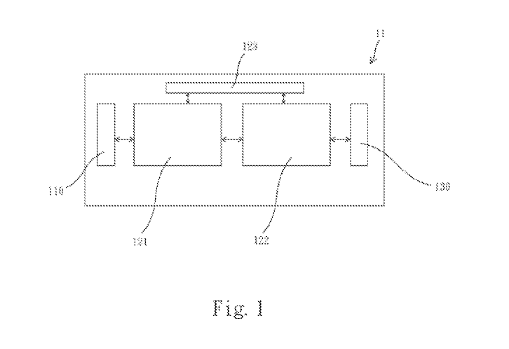

Please refer to FIG. 1, showing a heart rate variability device according to a preferred embodiment of the invention. In this embodiment, a heart rate variability device 11 may be held in a hand, in which a sensing element 110 is utilized to sense a heart beat signal of a pressure patient, a heart rate variability central processing unit (CPU) 121 is utilized to obtain parameters of heart rate variability, while proceeding with calculation, comparison and analysis with respect to the parameters of heart rate variability, it is to search for corresponding analysis data in a built-in tablet of a database in memory 123, and after completing test, it is connected to an internet and then to a cloud via a wireless telecommunication and communication module 122.

In this embodiment, the memory 123 of the heart rate variability device 11 is provided with a database. The database is stored with analysis data in multi-states after inducing and arranging and with a built-in tablet for looking up. The heart rate variability device 11 makes use of the sensing element 110 to receive the heart beat signal, and the heart beat signal is amplified, filtered, digitized, and converted and calculated to obtain a plurality of parameters of heart rate variability. The heart rate variability device 11 may comprise a first high pass filter, a first amplifier, a first low pass filter, a voltage/current converter, a comparator circuit, a second high pass filter, a photo-isolator and an antenna connected to a wireless communication module 130, but is not limited to these.

The wireless communication module 130 may be one of various wireless communication modules of 2.5G, 2.75G (GPRS), 3G (WCDMA, CDMA2000, or TD-CDMA) 3.5G and 4G (WIMAX, LTE, or TD-LTE) connected to a mobile phone base station, which is then connected to the cloud via the base station (not shown in the drawing). Of course, the heart rate variability device 11 may be wired and connected to a PC and then to the cloud.

After the heart rate variability device 11 obtaining the parameters of heart rate variability, it is to proceed with calculation, comparison and analysis with respect to the parameters of heart rate variability, and then to search for corresponding analysis data in the built-in tablet of the database in the heart rate variability device 11. In the same time, a display on the hand-held heart rate variability device 11 may display inspection data of the parameters of heart rate variability. Certainly, such inspection data may be printed out through connection to the computer.

In a preferred embodiment of the invention, the heart rate variability device 11 is a computer provided with capability of digital signal processing (DSP), while capable of proceeding with frequency-domain analysis, time-domain analysis and non-linear analysis.

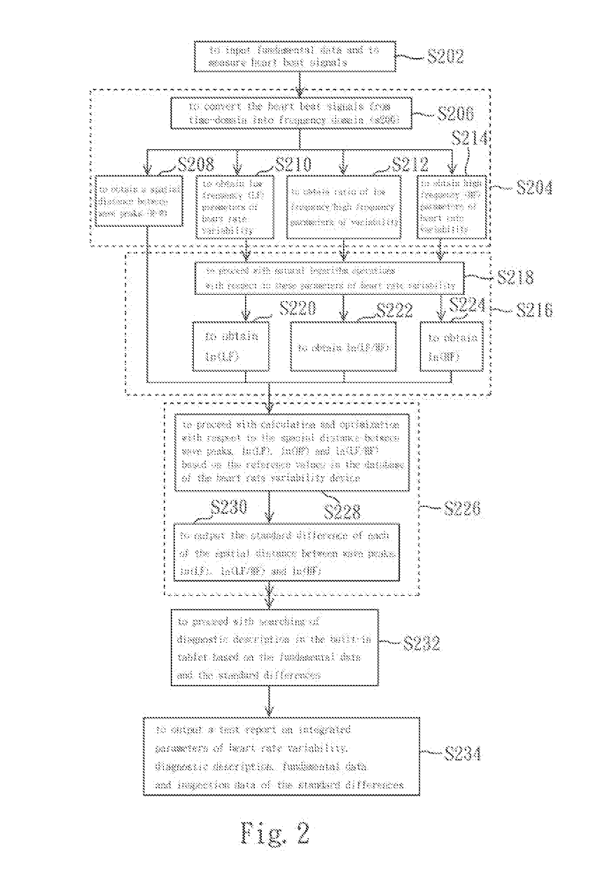

FIG. 2 is a flow chart showing analysis steps for an autonomic nerve according to a preferred embodiment of the invention. Such an analysis approach of the autonomic nerve makes diagnosis with respect to the autonomic nerve of a pressure patient in a non-invasive manner. In this embodiment, for example, it is to collect heart beat signals of the pressure patient in a couple of minutes.

In the analysis approach, it is first to input fundamental data of the pressure patient and to begin to measure the heart beat signals of the pressure patient (s202), in which the fundamental data of the pressure patient includes serial number, name, age, sex and website preference of the pressure patient, but not limited to these.

Subsequently, it is to proceed with conversion with respect to the received heart beat signals so as to obtain a plurality of parameters of heart rate variability (s204). The step s204 includes using Fast Fourier Transform to convert the heart beat signals from time-domain into frequency domain (s206), and obtaining thereafter the plurality of parameters of heart rate variability, such as a special distance between wave peaks (R-R) (s208), low frequency (LF) parameters of heart rate variability (s210), high frequency (HF) parameters of heart rate variability (s214), and low frequency/high frequency ratio of parameters of variability (s212).

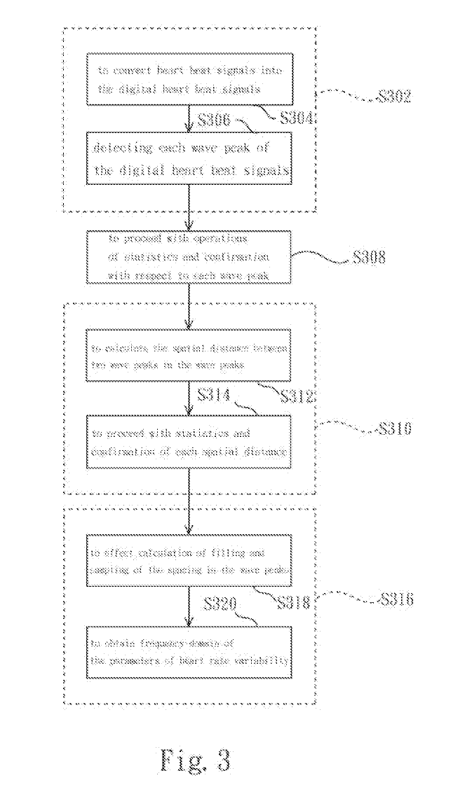

The detailed flow of step s204 for converting the received heart beat signals and obtaining a plurality of parameters of heart rate variability is shown in FIG. 3. Please refer to FIG. 3. The step s204 includes effecting digital conversion of the heart beat signals and detecting most wave peaks of the digital heart beat signals (s302).

Wherein, the proceeding of digitally converting the heart beat signals and detecting a plurality of wave peaks of the digital heart beat signals is to utilize the analog/digital converter in the heart rate variability device to convert the heart beat signals into the digital heart beat signals (s304). Then, the heart rate variability device detects each wave peak of the digital heart beat signals (s306).

In this embodiment, after detecting the wave peaks, it is to proceed with operations of statistics and confirmation with respect to each wave peak (s308). After that, the heart rate variability device effects calculating to obtain spacing between the most wave peaks in the wave peaks and effects statistics and confirmation of each spatial distance between two wave peaks in the wave peaks (s310). The heart rate variability device is to calculate the distance between two wave peaks in the wave peaks to obtain spacing of the most wave peaks (s312). After obtaining the special distance between the wave peaks, it is to proceed with statistics and confirmation of each spatial distance between two wave peaks in the wave peaks (s314).

Finally, the heart rate variability device effects calculating with respect to the wave peak spacing to obtain the frequency-domain of the parameters of heart rate variability (s316). The calculation of the special distance between wave peaks is directed to filling and sampling of the spacing in the wave peaks (s318), thereby obtaining the frequency-domain of the parameters of heart rate variability (s320).

Please continue to refer to FIG. 2. In this embodiment, after obtaining the parameters of heart rate variability, i.e. after step s204, the heart rate variability device effects natural logarithm operation with respect to at least one of the parameters of heart rate variability and obtains natural logarithm of the parameters of heart rate variability (s216). In the step s216, it is to proceed with natural logarithm operations with respect to LF parameters of heart rate variability, HF parameters of heart rate variability and the ratio of LF/HF parameters of heart rate variability (s218). Then, it will obtain the natural logarithm of LF parameters of heart rate variability ln(LF) (s220), the natural logarithm of HF parameters of heart rate variability ln(HF)(s222) and the natural logarithm of the ratio of LF/HF parameters of heart rate variability ln(LF/HF) (s224).

And then, it is to proceed with calculation and optimization with respect to the parameters of heart rate variability after operation based on most reference values in the database of the heart rate variability device and to output most standard difference obtained (s226). The proceeding of the calculation and optimization with respect to the parameters of heart rate variability after operation based on the most reference values in the database of the heart rate variability device and the proceeding of outputting the obtained most standard difference are achieved by utilizing artificial intelligence to proceed with calculation and optimization with respect to the special distance between wave peaks, ln(LF), ln(HF) and ln(LF/HF) based on the reference values in the database of the heart rate variability device (s228) and then it is to output the standard difference of each of the special distance between wave peaks, ln(LF), ln(HF) and ln(LF/HF) (s230).

In this embodiment, after obtaining the standard difference of each of the spacing between wave peaks, ln(LF), ln(HF) and ln(LF/HF), it is to proceed with searching of consistent analysis data in the built-in tablet based on the fundamental data of the pressure patient and the standard differences (s232).

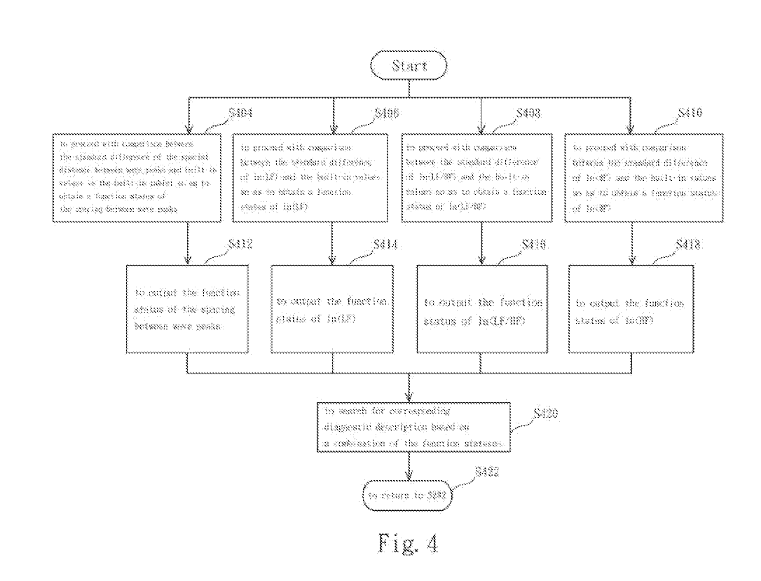

In the step s232, the operation flow of searching of consistent analysis data in the built-in tablet based on the fundamental data of the pressure patient and the standard differences is shown in FIG. 4. Please refer to FIG. 4. In the heart rate variability device, it is respectively to proceed with comparison between the standard difference of the special distance between wave peaks and the most built-in values in the built-in tablet so as to obtain a function status of the spacing between wave peaks (s404), to proceed with comparison between the standard difference of ln(LF) and the most built-in values in the built-in tablet so as to obtain a function status of ln(LF) (s406), to proceed with comparison between the standard difference of ln(LF/HF) and the most built-in values in the built-in tablet so as to obtain a function status of ln(LF/HF) (s408), and to proceed with comparison between the standard difference of ln(HF) and the most built-in values in the built-in tablet so as to obtain a function status of ln(HF) (s410). Subsequently, it is to respectively output the function status of the spacing between wave peaks (s412), the function status of ln(LF) (s414), the function status of ln(LF/HF) (s416) and the function status of ln(HF) (s418). Lastly, it is to search for the corresponding analysis data in the built-in tablet based on a combination of the function statuses (s420). Thereafter, it is to return to s232 (s422).

Each of the function statuses includes, for example, three statuses, i.e. L (low), N (middle) and H (high). Thus, the total number of combination of the function statuses of the parameters of heart rate variability will include 3×3×3×3=81.

Please refer to FIG. 2. In this embodiment, in the last, it is to output the integrated parameters of heart rate variability, analysis data, fundamental data and inspection data of the standard differences (s234).

In the preferred embodiment of the invention, the analysis method of the autonomic nerve includes: measuring an R wave of electric waves of a palm of the pressure patient, inputting to the signal amplifier a weak signal thereof sensed by the sensing element, filtering out from a lot of noises to obtain an ORS wave and amplifying the same, and converting the analog signal into the digital signal via the analog/digital converter.

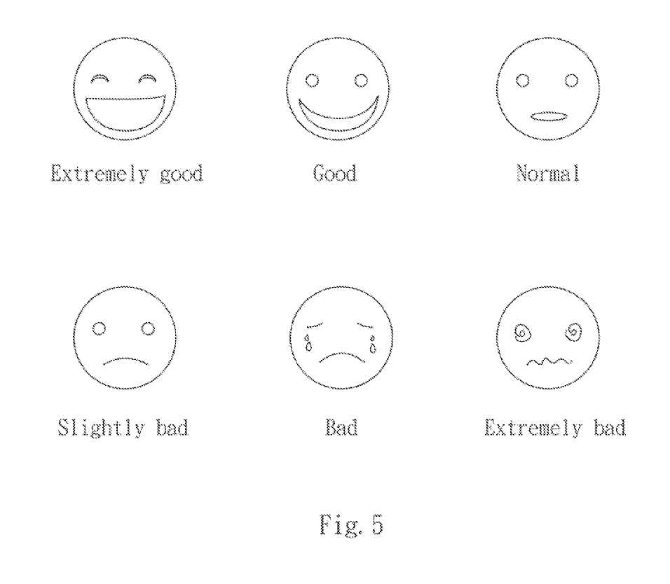

Pressure states of the pressure patient revealed in the inspection data are shown in a state diagram of FIG. 5. The pressure states include six kinds, being very good, good, normal, slightly bad, bad and very good.

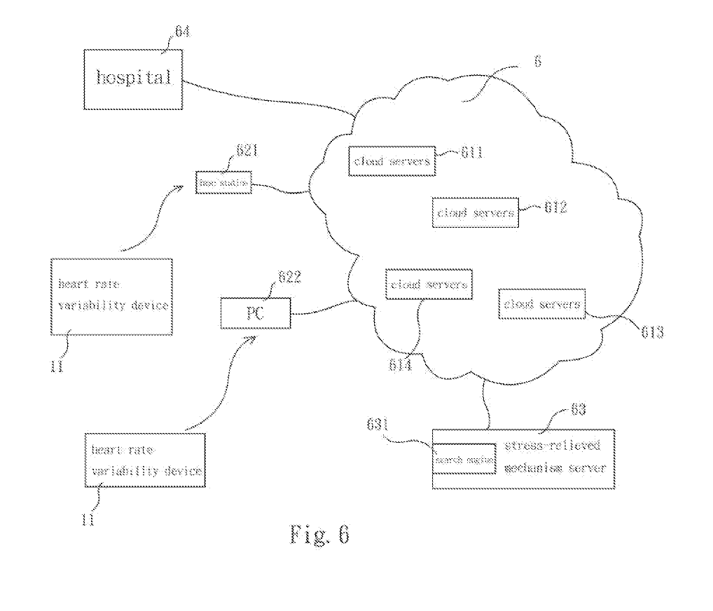

FIG. 6 shows a cloud health management system for heart rate variability according to a preferred embodiment of the invention. A symptom data package formed by: inspection data of measuring the autonomic nerve by a heart rate variability device held 11 by a pressure patient A, date, parameters of heart rate variability, analysis data, fundamental data and inspection data of standard differences, is transferred to cloud servers 611,612,613,614 for storing symptom data of the pressure patient via a base station 621 or PC 622, while a display of the heart rate variability device held 11 will present on its right side a hyperlink to a stress-relieved mechanism 63 preferred by the pressure patient through a search engine 631 of the stress-relieved mechanism 63. In this time, the stress-relieved mechanism is a music website 71, such as KKBOX, i.e. listening music to reduce pressure after connecting to the music website. On the other hand, another symptom data package (for example, 2.75G communication standard) formed by: inspection data of measuring the autonomic nerve by the heart rate variability device held 11 by another pressure patient B, date, parameters of heart rate variability, analysis data, fundamental data and inspection data of standard differences, is transferred to the cloud servers 611,612,613,614 for storing the symptom data of the pressure patient B via the base station 621 or PC 622, while the display of the heart rate variability device held 11 will present on its right side another hyperlink to a stress-relieved mechanism 63 preferred by the pressure patient B through the search engine 631 of the stress-relieved mechanism 63 in dependence of the extent of pressure, such as in a situation that it requires to seek opinions of a doctor if the pressure patient is in a state of extremely bad for certain days. In this situation, the stress-relieved mechanism is SPA. After connecting to the SPA website, it may use a serial number of the patient to make reservation for SPA to proceed with SPA for stress-relieving. After a couple of days, the heart rate variability device and the cloud health managing system of the invention may build a medical record information bank for storing symptom data of many pressure patient via the cloud so as to facilitate a heart science subjects doctor or family medical department doctor of a hospital 64 to check stress medical record information of a stress patient for remote medical use. In the meantime, a son of a pressure patient may look up the stress medical record information of his parent(s) through the heart rate variability device after obtaining an encrypted code or account number of the medical record information bank of his parent(s).

The stress-relieved mechanism may be a stress-relieved music mechanism, yoga mechanism, imperial sacrifices sit mechanism, mind traveling mechanism, hot spring malingers mechanism, game software mechanism, Chinese shadow boxing mechanism, forward ponder learning mechanism, essential oil stress-relieved mechanism, stress-relieved massager mechanism, meditation courses mechanism, rhythm breathing mechanism, Brocade qigong mechanism or stress-relieved food and drink restaurant.

The stress-relieved mechanism is developed and incorporated with the inventor and the telecommunication industry (such as China Telecommunication Co., Taiwan Mobile Phone Co. and Far Biography telecommunication Co.) under a cloud business model, including money stream and message stream. For example, payment will adopt membership for a member. It may present on the display 70 the medical record information measured by the heart rate variability device 11, while collecting fees to the pressure patient through the telecommunication industry.

The message stream includes an update news 721 for stress-relieving, information of mental healing, and information and websites of the stress-relieved mechanisms in the world. Such message stream will be stored in the server(s) of the stress-relieved mechanism(s).

Although the present invention has been explained in relation to its preferred embodiments, it is to be understood that other possible modifications and variations can be made without departing from the scope of the invention as claimed below.

We claim:

1. A heart rate variability device, comprising:
a sensing element for sensing a heart beat signal of a pressure patient;
a signal processing module having a first high pass filter, a first amplifier, a first low pass filter, a voltage/current converter, a comparator circuit, a second high pass filter and an analog/digital converter, for converting the heart beat signal into an autonomic nerve output signal;
a heart rate variability central processing unit (CPU), being installed with a heart rate variability processing software for outputting inspection data after processing and operating the autonomic nerve output signal; and
a wireless telecommunication and communication module for receiving the inspection data and transferring the inspection data to an internet access device via wireless communication.

2. A heart rate variability device of claim 1, wherein the internet access device is a base station or a PC.

3. A heart rate variability device of claim 1, wherein the wireless telecommunication and communication module adopts communication standard of 2.75G, 3G, 3.5G or 4G.

4. A cloud health managing system of heart rate variability frequency spectrum, comprising:
more than one inspection data input/output device for inputting or outputting more than one inspection data of a pressure patient;
more than one internet access device, being connected to the inspection data input/output device for transferring the inspection data to a cloud via the internet access device;
more than one cloud server, being connected to the internet access device for storing the inspection data, and making use of the inspection data input/output device for accessing the inspection data; and
a stress-relieved mechanism server, being connected to the cloud server and having a search engine, in which as a pressure index in the inspection data exceeds a preset value, a hyperlink of a stress-relieved mechanism is presented on the inspection data input/output device so as to connect to a website of the stress-relieved mechanism to facilitate proceeding of stress-relieving of the pressure patient.

5. A cloud health managing system of heart rate variability spectrum of claim 4, wherein the inspection data input/output device is a computer or a heart rate variability device as claimed in claim 1.

6. A cloud health managing system of heart rate variability spectrum of claim 4, wherein the stress-relieved mechanism is a stress-relieved music mechanism, yoga mechanism, imperial sacrifices sit mechanism, mind traveling mechanism, hot spring malingers mechanism, game software mechanism, Chinese shadow boxing mechanism, forward ponder learning mechanism, essential oil stress-relieved mechanism, stress-relieved massager mechanism, meditation courses mechanism, rhythm breathing mechanism, Brocade qigong mechanism or stress-relieved food and drink restaurant.
